(19) United States
(12) Patent Application Publication
(10) Pub. No.: US 2024/0166952 A1
(43) Pub. Date: May 23, 2024

(54) TWO STEP PROCESS FOR CHEMICALLY RECYCLING PLASTIC WASTE

(71) Applicant: Anellotech, Inc., Pearl River, NY (US)

(72) Inventors: Torren Carlson, Pearl River, NY (US); Leslaw Mleczko, Pearl River, NY (US); Omar M. Basha, Wilmington, DE (US); William Igoe, Pearl River, NY (US)

(21) Appl. No.: 17/990,642

(22) Filed: Nov. 18, 2022

(51) Int. Cl. C10G 1/00; B01D 21/26; B01J 8/24; B01J 29/40; C07C 4/06; C10G 1/10

(52) U.S. Cl. CPC C10G 1/002; B01D 21/267; B01J 8/24; B01J 29/40; C07C 4/06; C10G 1/10; B01J 2208/00805; C07C 2529/40; C10G 2300/1003; C10G 2400/20; C10G 2400/22; C10G 2400/30

(57) ABSTRACT

A two-step process is described that includes a pyrolytic first step and a catalytic fluid bed second step that without separation upgrades the resulting raw pyrolysis products, for the conversion of waste plastics, polymers, and other waste materials to useful chemical and fuel products such as paraffins, olefins, and aromatics such as BTX.

### FIELD OF THE INVENTION

This invention relates to the conversion of waste plastics, polymers, and other waste materials to useful chemical and fuel products such as paraffins, olefins, and aromatics such as BTX (a mixture of benzene, toluene, and xylenes) in a two-step process that includes a pyrolytic first step and a second step that upgrades the resulting product mixture.

### INTRODUCTION

In 2019, plastics generation in the United States was 55.2 million tons, which was 13 percent of MSW generation. World-wide over 368 million tons of plastics were produced. By some estimates, of the 8.3 billion tons of plastics ever produced, 6.3 billion tons ended up as waste, of which only 9% has been recycled. Plastic recycling recovers scrap or waste plastic and reprocesses the material into useful products. However, since China banned the import of waste plastics in 2018 the recycle rate in the US is estimated to have dropped to only 4.4%.

Plastic recycling is challenging due to the chemical nature of the long chain organic polymers and low economic returns. In addition, waste plastic materials often need sorting into the various plastic resin types, e.g. low density polyethylene (LDPE), high density polyethylene (HDPE), polypropylene (PP), polystyrene (PS), polyvinyl chloride (PVC), and polyethylene-terephthalate (PET) for separate recycling treatments. Pyrolytic and catalytic pyrolytic processes are known in which waste plastics are heated to produce products such as liquid oils, gases, and carbon black.

Plas-TCat™ is a catalytic fluid bed process using zeolite catalysts to convert polymer/plastic material, especially waste plastics that otherwise might be sent to a landfill or incinerator, to a mixed product of permanent gases, C2-C4 light olefins, C1-C4 light paraffins, and C5+ hydrocarbons including benzene, toluene, and xylenes (“BTX”), aromatic and non-aromatic naphtha range molecules, C11+ hydrocarbons, coke and char, and minor byproducts. Plastic mixtures that have relatively high hydrogen to carbon molar ratio, such as polyethylene (PE), polypropylene, polystyrene, and combinations thereof, can be converted to olefins and aromatics.

U.S. Pat. Appl. No. 2016/0289569 from Baird et al describes a process of pyrolyzing biomass to bio-oil, separating and upgrading the pyrolysis oil, deoxygenating the upgraded pyrolysis oil to obtain aromatic and paraffinic products, and further upgrading the paraffinic product by aromatization.

U.S. Pat. No. 10,233,395 to Ward relates to a process for converting mixed waste plastic (MWP) into petrochemicals wherein a MWP stream is fed to a pyrolysis reactor, converting said MWP into separated gaseous stream and liquid streams, and further separately processing the gaseous stream and the liquid stream.

Fukuda et al in U.S. Pat. No. 4,851,601, describe a process for pyrolyzing plastics in a tank reactor with an added solid to minimize materials sticking to the reactor wall, and reacting the vapors in a fixed bed catalytic reactor.

Bartek et al in U.S. Pat. No. 9,040,761 describe a process for pyrolyzing biomass and plastic in a fluidized bed of heat transfer material and reacting the products with a catalyst in a second reactor to produce a bio-oil.

Saito et al in U.S. Pat. No. 4,584,421 describe a process for melting and thermally decomposing plastic scraps with heat and passing the vaporous product through a bed of catalyst particles.

Barbarias et al in “Catalyst Performance in the HDPE Pyrolysis-Reforming Under Reaction-Regeneration Cycle,” Catalysts 2019, 9, 414, and “Waste Plastics Valorization by Fast Pyrolysis and in Line Catalytic Steam Reforming for Hydrogen Production,” in M. Olazar Materials Science, DOI:10.5772/INTECHOPEN.85048, 9 Jul. 2019, describe the conversion of waste plastic to syngas by thermal pyrolysis in a conical spouted bed reactor and steam reforming of the volatiles formed (gas and waxes) in a fluid bed of Ni-containing catalyst.

Schenk et al in US Patent Application US20220195310A1 describe a process for preparing BTX by pyrolyzing a plastic mixture at 600-1000° C. and catalytically upgrading the vapors in a fluidized bed at 450-700° C. to a mixture comprising aromatics.

### SUMMARY

A method of producing olefinic and aromatic hydrocarbons from waste plastics comprising feeding a mixture of plastics to a two-stage process including a first stage in which the plastic mixture is pyrolyzed anaerobically and a second stage in which, without separation, the raw products of the first stage are catalytically reacted to produce olefins and aromatics.

In a first aspect of this invention a mixture comprising polymers is converted in an anaerobic process in a reactor to produce a pyrolyzed stream that is fed to a fluid bed catalytic pyrolysis process to produce olefins and aromatics.

In another aspect, the invention provides a method of converting plastics to olefins, or aromatics, or a mixture of olefins and aromatics, comprising: feeding a polymer or mixture of polymers to a first pyrolysis reactor; anaerobically pyrolyzing the stream in the first reactor under conditions sufficient to produce a raw product mixture comprising one or more olefins and paraffins; without separation transferring the raw product mixture from the first pyrolysis reactor to a fluidized bed catalytic reactor where, in the presence of a catalyst, the mixture is converted to a product vapor mixture; and recovering olefins or aromatics or some combination thereof from the product vapor mixture.

The inventive method may be further characterized by one or any combination of the following features:
feeding a feed mixture comprising plastics to a pyrolysis reactor; wherein the feed mixture comprises plastics chosen from among polyethylene, polypropylene, polyesters, polyethylene terephthalate (PET), acrylonitrile-butadiene-styrene (ABS) copolymers, polyamide, polyurethane, polyethers, polycarbonates, poly(oxides), poly(sulfides), polyarylates, polyetherketones, polyetherimides, polysulfones, polyurethanes, polyvinyl alcohols, and polymers produced by polymerization of monomers, such as, for example, dienes, olefins, styrenes, acrylates, acrylonitrile, methacrylates, methacrylonitrile, diacids and diols, lactones, diacids and diamines, lactams, vinyl esters, block copolymers thereof, and alloys thereof; thermoset polymers such as, for example, epoxy resins; phenolic resins; melamine resins; alkyd resins; vinyl ester resins; unsaturated polyester resins; crosslinked polyurethanes; polyisocyanurates; crosslinked elastomers, including but not limited to, polyisoprene, polybutadiene, styrene-butadiene, styrene-isoprene, ethylene-propylene-diene monomer polymer; and mixtures thereof, wherein the feedstock comprises a mix of waste plastic chosen from among polyethylene terephthalate (PET), high density polyethylene (HDPE), polyvinyl chloride (PVC) or polyvinylidene (PVCD), low density polyethylene (LDPE), polypropylene (PP), polystyrene (PS), or mixed resins, or some combination thereof;
wherein the feed stream of plastics is first heated to at least 200° C. in a thermal treatment reactor to achieve a molten state and filtered to remove solids; wherein the feed mixture is heated anaerobically to a temperature of between 250 and 300° C. to at least partially decompose the polymers in the pyrolysis reactor;
wherein the thermal treatment or pyrolysis reactor is one or more moving bed, 1-screw extruder, two screw extruder, auger reactor, rotating kiln reactor, or a stepped grate reactor, or some combination thereof; wherein the first pyrolysis reactor comprises and inlet port and an exit port, the temperatures can be from 20 to 225° C., such as 20 to 100° C., or 20 to 50° C., at or near the inlet port, and the range of temperatures at the high temperature exit port can be from 300 to 700° C., such as from 325 to 650° C., from 350 to 600° C., or from 350 to 575° C.;
wherein the residence time of condensed phases in the first pyrolysis reactor, or in either reactor when there are more than one pyrolysis reactors (prior to the catalytic fluidized bed reactor), is at least 1, or at least 5, or at least 10, or at least 20, or at least 30, or from 1 to 60, or from 5 to 30, or from 10 to 30 minutes;
wherein the pyrolysis reactor comprises two or more reactors in series;
wherein the feed is heated to a temperature between 250 and 300° C. in the first pyrolysis reactor and the products are passed to a second pyrolysis reactor;
wherein a sweep gas such as H2O, N2, Ar, CO2, or some combination thereof, is fed to the thermal treatment reactor and the vapors are exhausted;
wherein the non-vapor products of the catalytic pyrolysis reactor, or a portion of the gases remaining after removal of desired products, or both, are combusted to provide energy for the pyrolysis or catalytic pyrolysis process;
wherein a solid co-reactant material is fed to a thermal treatment reactor;
wherein the solid co-reactant comprises one or more materials chosen from among agricultural lime, calcium oxide, calcium hydroxide, magnesium oxide, magnesium hydroxide, limestone, hydrotalcites, activated carbon, or zeolite, or other solid basic material, or some combination thereof;
wherein the solid co-reactant material is transferred to a combustion regenerator wherein the carbonaceous materials are reacted with air and at least a portion of the hot solid co-reactant material is returned to the thermal treatment reactor;
wherein the products produced in the pyrolysis reactor are transferred, without separating a significant portion of the products, to a catalytic pyrolysis reactor containing a catalyst;
wherein a vapor phase co-reactant comprising H2, CO, or olefins, or some combination of these, or a recycle stream is fed directly to the pyrolysis reactor or the catalytic pyrolysis reactor;
wherein a portion of the vapor products from the catalytic pyrolysis process is fed to a condenser where it is cooled to produce condensed materials;
wherein a portion of the condensed material separated from the product stream from the pyrolysis or the catalytic pyrolysis reactor is separated into fractions and at least a portion is recycled to the pyrolysis reactor;
wherein the condensed material is separated into fractions by distillation and at least a portion of the paraffins, olefins, or aromatics or their combination that contain more than 7 carbon atoms is recycled to the pyrolysis or catalytic pyrolysis reactor;
wherein the condensed material separated from the product stream from the pyrolysis or the catalytic pyrolysis reactor the condensed material is separated into fractions by distillation and at least a portion of the fraction boiling above 300° C. or boiling in the range 300 to 800° C. is recycled to the pyrolysis or catalytic pyrolysis reactor;
wherein the catalytic reactor is a fluidized bed reactor;
wherein the catalytic reaction is conducted in a fluidized bed, circulating bed, bubbling bed, or riser reactor at an operating temperature in the range from 300° C. to 800° C., from 350° C. to 750° C., from 400° C. to 700° C., from 450° C. to 650° C., from 500° C. to 600° C., or from 525° C. to 575° C.;
wherein the pressure is at least 0.1 MPa (1 bar), at least 0.3 MPa (3 bar), or at least 0.4 MPa (4 bar), or from 0.1 to 2.0 MPa (1 to 20 bar), from 0.1 to 1.0 MPa (1 to 10 bar), or from 0.3 to 0.8 MPa (3 to 8 bar), preferably from 0.4 to 0.6 MPa (4 to 6 bar);
wherein the fluidization gas for the catalytic pyrolysis can comprise H2, CO, CO2, H2O, C1-C4 paraffins or olefins or both, N2, Ar, He, or a recycle stream, or some combination thereof;
wherein the residence time of the fluidization gas in the catalytic pyrolysis reactor, defined as the volume of the reactor divided by the volumetric flow rate of the fluidization fluid under process conditions of temperature and pressure, can be from 1 second to 480 seconds, or from 1 second to 240 seconds, or from 2 seconds to 60 seconds, or from 3 seconds to 30 seconds, or from 4 seconds to 15 seconds;
wherein the catalyst is a solid catalyst and the step of catalytically pyrolyzing comprises pyrolyzing in the presence of the solid catalyst in a fluidized bed reactor to produce a fluid product stream and used catalyst with coke, and wherein at least 95% the carbon in the feed is converted to coke and volatile products;
wherein the catalyst comprises a zeolite;
wherein the catalyst may be selected from naturally occurring zeolites, synthetic zeolites and combinations thereof;
wherein the catalyst may be selected from ZSM-5, ZSM-11, ZSM-12, ZSM-22, ZSM-23, ZSM-35, ZSM-48, ZSM-50, or combinations thereof;
wherein the catalyst comprises ZSM-5;
wherein the product vapor mixture from the catalytic conversion comprises at least 20 mass % olefins, or at least 50 mass % olefins, in some embodiments in the range of 20 to 90 mass % olefins;
wherein the catalyst composition comprises a crystalline molecular sieve characterized by an SAR from greater than 12 to 240 and a CI from 5 to 10;
wherein the mass yield of olefins in the product vapor mixture from the catalytic conversion is at least 30%, or at least 40%, or at least 45%, or at least 50%, or at least 55%, or at least 60%, or from 20% to 90%, or from 30% to 70%, or from 45% to 60%, olefins based on the mass in the polymer feed;
wherein the mass yield of BTX in the gaseous product mixture from the catalytic conversion is at least 30%, or at least 40%, or at least 45%, or at least 50%, or at least 55%, or at least 60%, or from 20% to 90%, or from 30% to 70%, or from 45% to 60% BTX based on the mass in the polymer feed;
wherein the vapor products of the catalytic pyrolysis are passed through one or more solids separation devices comprising a cyclone;
wherein catalyst in the catalytic pyrolysis reactor is withdrawn and regenerated by oxidation with air, and returned to the catalytic pyrolysis reactor;
wherein heat from the regeneration of the catalyst provides energy to the step of thermal treatment or pyrolyzing;
wherein at least a portion of the gases in the product mixture are combusted in the regenerator;
wherein the gaseous catalytic pyrolysis product mixture is subjected to a separation process to produce a stream of gases enriched in CH₄, CO, and H₂; and passing at least a portion of the stream of gases enriched in CH₄, CO, and H₂ to the regenerator where they are combusted;
wherein the gaseous catalytic pyrolysis product mixture comprises CH4 and C2-C4 paraffins;
wherein 50 to 100 mass % of the CH₄ and C2-C4 paraffins is combusted in the regenerator;
wherein at least 2, or at least 3, or at least 5, or at least 7, or at least 10, or at least 15, or from 2 to 20, or from 3 to 10, or from 5 to 10 plastics upgrading facilities, i.e. pyrolysis and catalytic pyrolysis units, feed a central separation and purification facility;
wherein the total crude product mixture prepared at the at least 2 plastics upgrading facilities that is introduced into a central separation and purification facility is at least 20, or at least 50, or at least 100, or at least 150, or at least 200 metric tons per day, or from 20 to 500, or from 30 to 200, or from 50 to 150 metric tons per day of crude product mixture;
wherein the central facility for separation and purification is at a refinery; and
wherein benzene, toluene, xylenes, p-xylene, m-xylene, o-xylene, BTX (a mixture of benzene, toluene, and xylenes), C6-C20 paraffins and olefins, ethylene, propylene, or naphthalene, or others, or some combination of these prepared from waste plastics at more than one upgrading facility, is separated and purified at a central separation and purification facility.

There are many advantages of chemically recycling plastics by pyrolysis in a thermochemical reactor including: a mixture of any type of plastics is suitable, the plastic particles need not be ground to small size since the long residence time in the pyrolysis reactor or reactors ensures that the plastic pieces are heated to decomposition temperatures, the pyrolysis can be operated at high temperatures, and undesirable contaminants can be removed in an optional thermal treatment reactor.

Advantages of a two-step plastics upgrading process include: simple feeding system for the raw pyrolysis product to the catalytic step, no danger of agglomeration in the fluidized bed causing defluidization or clumping in the bed, no need for good mixing of solid or molten plastic feed with catalyst particles, significantly narrower residence time distribution for pyrolysis gas in the catalytic reactor compared with the feed of solids, thus resulting in fewer heavy products, no carryover of plastic particles into the catalyst regenerator, fewer external impurities transferred into the catalytic reactor and no inorganic particles embedded in the polymer like fillers or additives are transferred into the catalytic reactor when an optional thermal treatment reactor is used (fillers are usually alkaline (basic) and when reacting with the acidic catalyst cause its deactivation), heat is convectively supplied to the plastics without the use of steam that causes deactivation (dealumination) of the catalyst, and a greatly reduced need for additional fluidization gas rendering product recovery simpler and less costly.

Another advantage of the two step plastics upgrading process is that the production of a crude liquid product stream made from recycled plastic by the inventive process can be conducted at a separate location from the product separation and purification system, and this “distributed processing” scheme minimizes the costs of separation and purification for small scale regional plastics upgrading facilities

### BRIEF DESCRIPTION OF THE DRAWINGS

FIG. 1 schematically illustrates a process for converting mixed plastic materials to valuable products by pyrolyzing the mixed plastics and catalytically reacting the raw product mixture to produce olefins, aromatics, or some combination thereof.

FIG. 2 schematically illustrates a process for converting mixed plastic materials to valuable products by pyrolyzing the mixed plastics and catalytically reacting the products to produce olefins, aromatics, or some combination thereof, wherein a thermal treatment reactor is used to remove contaminants before feeding the pyrolysis reactor.

FIG. 3 schematically illustrates an embodiment of the invention in which five plastics upgrading units are connected to feed a single product separation and purification facility in a hub and spoke system.

FIG. 4 shows a drawing of the reactor used in the Examples.

### GLOSSARY

Aromatics—As used herein, the terms “aromatics” or “aromatic compound” are used to refer to a hydrocarbon compound or compounds comprising one or more aromatic groups such as, for example, single aromatic ring systems (e.g., benzyl, phenyl, etc.) and fused polycyclic aromatic ring systems (e.g., naphthyl, 1,2,3,4-tetrahydronaphthyl, etc.). Examples of aromatic compounds include, but are not limited to, benzene, toluene, indane, indene, 2-ethyl toluene, 3-ethyl toluene, 4-ethyl toluene, trimethyl benzene (e.g., 1,3,5-trimethyl benzene, 1,2,4-trimethyl benzene, 1,2,3-trimethyl benzene, etc.), ethylbenzene, styrene, cumene, methylbenzene, propylbenzene, xylenes (e.g., p-xylene, m-xylene, o-xylene, etc.), naphthalene, methyl-naphthalene (e.g., 1-methyl naphthalene, anthracene, 9.10-dimethylanthracene, pyrene, phenanthrene, dimethyl-naphthalene (e.g., 1,5-dimethylnaphthalene, 1,6-dimethylnaphthalene, 2,5-dimethylnaphthalene, etc.), ethyl-naphthalene, hydrindene, methyl-hydrindene, and dymethyl-hydrindene. Single-ring and/or higher ring aromatics may also be produced in some embodiments.

Fluid—The term “fluid” refers to a gas, a liquid, a mixture of a gas and a liquid, or a gas or a liquid containing dispersed solids, liquid droplets and/or gaseous bubbles. The terms “gas” and “vapor” have the same meaning and are sometimes used interchangeably. In some embodiments, it may be advantageous to control the residence time of the fluidization fluid in the reactor. The fluidization residence time of the fluidization fluid is defined as the volume of the reactor divided by the volumetric flow rate of the fluidization fluid under process conditions of temperature and pressure.

Fluidized Bed Reactor—The term “fluidized bed reactor” is given its conventional meaning in the art and is used to refer to reactors comprising a vessel that can contain a granular solid material (e.g., silica particles, catalyst particles, etc.), in which a fluid (e.g., a gas or a liquid) is passed through the granular solid material at velocities sufficiently high as to suspend the solid material and cause it to behave as though it were a fluid. Examples of fluidized bed reactors are described in “Fluidization Engineering” by D. Kunii and O. Levenspiel, Butterworth-Heinemann, 1991, incorporated herein by reference. The term “circulating fluidized bed reactor” is also given its conventional meaning in the art and is used to refer to fluidized bed reactors in which the granular solid material is passed out of the reactor, circulated through a line in fluid communication with the reactor, and recycled back into the reactor. Examples of circulating fluidized bed reactors are described in “Fluidization Engineering” by D. Kunii and O. Levenspiel, Butterworth-Heinemann, 1991.

Bubbling fluidized bed reactors and turbulent fluidized bed reactors are also known to those skilled in the art. In bubbling fluidized bed reactors, the fluid stream used to fluidize the granular solid material is operated at a sufficiently low flow rate such that bubbles and voids are observed within the volume of the fluidized bed during operation. In turbulent fluidized bed reactors, the flow rate of the fluidizing stream is higher than that employed in a bubbling fluidized bed reactor, and hence, bubbles and voids are not observed within the volume of the fluidized bed during operation. Examples of bubbling and turbulent fluidized bed reactors are described in Kirk-Othmer Encyclopedia of Chemical Technology (online), Vol. 11, Hoboken, N.J.: Wiley-Interscience, 2001, pages 791-825, incorporated herein by reference.

Olefins—The terms “olefin” or “olefin compound” (a.k.a. “alkenes”) are given their ordinary meaning in the art and are used to refer to any unsaturated hydrocarbon containing one or more pairs of carbon atoms linked by a double bond. Olefins include both cyclic and acyclic (aliphatic) olefins, in which the double bond is located between carbon atoms forming part of a cyclic (closed-ring) or of an open-chain grouping, respectively. In addition, olefins may include any suitable number of double bonds (e.g., monoolefins, diolefins, triolefins, etc.). Examples of olefin compounds include, but are not limited to, ethene, propene, allene (propadiene), 1-butene, 2-butene, isobutene (2 methyl propene), butadiene, and isoprene, among others. Examples of cyclic olefins include cyclopentene, cyclohexane, cycloheptene, among others. Aromatic compounds such as toluene are not considered olefins; however, olefins that include aromatic moieties are considered olefins, for example, benzyl acrylate or styrene.

Catalysts—Catalyst components useful in the context of this invention can be selected from any catalyst known in the art, or as would be understood by those skilled in the art. Catalysts promote and/or affect reactions. Thus, as used herein, catalysts lower the activation energy (increase the rate) of a chemical process, and/or improve the distribution of products or intermediates in a chemical reaction (for example, a shape selective catalyst). Examples of reactions that can be catalyzed include: dehydration, dehydrogenation, isomerization, hydrogen transfer, hydrogenation, polymerization, cyclization, desulfurization, denitrogenation, deoxygenation, aromatization, decarbonylation, decarboxylation, aldol condensation, and combinations thereof. Catalyst components can be considered acidic, neutral, or basic, as would be understood by those skilled in the art.

For catalytic pyrolysis, particularly advantageous catalysts include those containing internal porosity selected according to pore size (e.g., mesoporous and pore sizes typically associated with zeolites), e.g., average pore sizes of less than about 10 nm, less than about 5 nm, less than about 2 nm, less than about 1 nm, less than about 0.5 nm, or smaller. In some embodiments, catalysts with average pore sizes of from about 0.5 nm to about 10 nm may be used. In some embodiments, catalysts with average pore sizes of between about 0.55 nm and about 0.65 nm, or between about 0.59 nm and about 0.63 nm may be used. In some cases, catalysts with average pore sizes of between about 0.7 nm and about 0.8 nm, or between about 0.72 nm and about 0.78 nm may be used.

In some preferred embodiments of catalytic pyrolysis, the catalyst may be selected from naturally occurring zeolites, synthetic zeolites and combinations thereof. In certain embodiments, the catalyst may be a ZSM-5 zeolite catalyst, as would be understood by those skilled in the art. Optionally, such a catalyst can comprise acidic sites. Other types of zeolite catalysts include: ferrierite, zeolite Y, zeolite beta, mordenite, MCM-22, ZSM-23, ZSM-57, SUZ-4, EU-1, ZSM-11, (S)AlPO-31, SSZ-23, among others. Zeolites and other small pore materials are often characterized by their Constraint Index. The Constraint Index approximates the ratio of the cracking rate constants for normal hexane and 3-methylpentane. The method by which Constraint Index is determined is described more fully in U.S. Pat. No. 4,029,716, incorporated by reference for details of the method.

Constraint Index (CI) values for some typical materials are:

TABLE 1
Constraint Indices of some common zeolites.

| Material | Constraint Index | Test Temp, ° C. |
|---|---|---|
| ZSM-4 | 0.5 | 316 |
| ZSM-5 | 6-8.3 | 371-316 |
| ZSM-11 | 5-8.7 | 371-316 |
| ZSM-12 | 2.3 | 316 |
| ZSM-20 | 0.5 | 371 |
| ZSM-22 | 7.3 | 427 |
| ZSM-23 | 9.1 | 427 |
| ZSM-34 | 50 | 371 |
| ZSM-35 | 4.5 | 454 |
| ZSM-48 | 3.5 | 538 |
| ZSM-50 | 2.1 | 427 |
| Mordenite | 0.5 | 316 |
| REY | 0.4 | 316 |
| Dealuminized Y | 0.5 | 510 |
| Beta | 0.6-2 | 316-399 |

The CI may vary within the indicated range of 1 to 12. Likewise, other variables such as crystal size or the presence of possibly occluded contaminants and binders intimately combined with the crystal may affect the CI. It is understood to those skilled in the art that the CI, as utilized herein, while affording a highly useful means for characterizing the molecular sieves of interest is approximate, taking into consideration the manner of its determination, with the possibility, in some instances, of compounding variable extremes. However, the CI will have a value for any given molecular sieve useful herein within the approximate range of 1 to 12.

In other embodiments, non-zeolite catalysts may be used; for example, WOx/ZrO2, aluminum phosphates, etc. In some embodiments, the catalyst may comprise a metal and/or a metal oxide. Suitable metals and/or oxides include, for example, nickel, palladium, platinum, titanium, vanadium, chromium, manganese, iron, cobalt, zinc, copper, gallium, and/or any of their oxides, among others. In some cases promoter elements chosen from among the rare earth elements, i.e., elements 57-71, cerium, zirconium or their oxides for combinations of these may be included to modify activity or structure of the catalyst. In addition, in some cases, properties of the catalysts (e.g., pore structure, type and/or number of acid sites, etc.) may be chosen to selectively produce a desired product.

Catalysts for other processes, such as alkylation of olefins, aromatization (hydrocarbon reforming), hydrogenation, hydrotreating, deoxygenation, denitrogenation, and desulfurization are well-known and can be selected for the olefin conversion or other processes described herein.

Plastics or Polymers—The terms “plastics” and “polymers” are used interchangeably herein. A polymer is a carbon-based (at least 50 mass % C) material chiefly made up of repeating units and having a number average molecular weight of at least 100, typically greater than 1000 or greater than 10,000. Polymers include thermoplastic polymers such as, for example, polyethylene, polypropylene, polyesters, polyethylene terephthalate (PET), acrylonitrile-butadiene-styrene (ABS) copolymers, polyamide, polyurethane, polyethers, polycarbonates, poly(oxides), poly(sulfides), polyarylates, polyetherketones, polyetherimides, polysulfones, polyurethanes, polyvinyl alcohols, and polymers produced by polymerization of monomers, such as, for example, dienes, olefins, styrenes, acrylates, acrylonitrile, methacrylates, methacrylonitrile, diacids and diols, lactones, diacids and diamines, lactams, vinyl halides, vinyl esters, block copolymers thereof, and alloys thereof, thermoset polymers such as, for example, epoxy resins; phenolic resins; melamine resins; alkyd resins; vinyl ester resins; unsaturated polyester resins; crosslinked polyurethanes; polyisocyanurates; crosslinked elastomers, including but not limited to, polyisoprene, polybutadiene, styrene-butadiene, styrene-isoprene, ethylene-propylene-diene monomer polymer; and blends thereof. Mixtures of polymers separated from municipal solid waste or other waste streams are suitable feeds provided they contain only small fractions of contaminants such as S, N, O, halogens, minerals, metals, or carbon black. Polymers yielding halogenated material upon pyrolysis, for example, polyvinyl chloride (PVC), polytetrafluoroethylene (PTFE), and other halogenated polymers, are generally minimized or excluded from the feed materials useful in this invention.

Pyrolysis—The terms “pyrolysis” and “pyrolyzing” are given their conventional meaning in the art and are used to refer to the transformation of a compound, e.g., a solid hydrocarbonaceous material, into one or more other substances, e.g., volatile organic compounds, gases and coke, by heat, preferably without the addition of, or in the absence of, O₂. Preferably, the volume fraction of O₂ present in a pyrolysis reaction chamber is 0.5% or less. Pyrolysis may take place with or without the use of a catalyst. “Catalytic pyrolysis” refers to pyrolysis performed in the presence of a catalyst and may involve steps as described in more detail below. Example of catalytic pyrolysis processes are outlined, for example, in Huber, G. W. et al, “Synthesis of Transportation Fuels from Biomass: Chemistry, Catalysts, and Engineering,” Chem. Rev. 106, (2006), pp. 4044-4098.

“Thermal treatment” is used herein as a process for heating a feed mixture to modest temperature at which some contaminants such as HCl, H2S, NH3 are evolved and can be exhausted, and the feed mixture becomes molten so that solids such as minerals, metals, and carbon black can be removed by filtration.

As is standard patent terminology, the term “comprising” means “including” and does not exclude additional components. Any of the inventive aspects described in conjunction with the term “comprising” also include narrower embodiments in which the term “comprising” is replaced by the narrower terms “consisting essentially of” or “consisting of.” As used in this specification, the terms “includes” or “including” should not be read as limiting the invention but, rather, listing exemplary components.

### DETAILED DESCRIPTION OF THE INVENTION

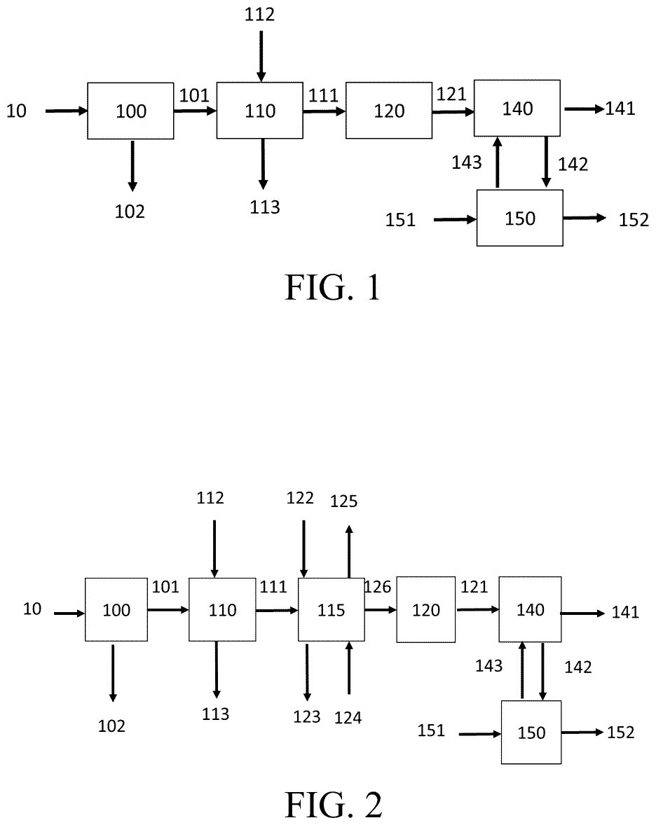

FIG. 1 presents a schematic of one embodiment of the inventive process for converting plastic waste to olefins and aromatics. A mixture of plastics 10 is introduced into an optional feed system 100 that prepares the plastic mixture for introduction into the process by, for example, removing undesirable feed materials 102 such as metal, minerals, halogenated materials, and the like, or sizing the material to the desired size range, or both. The steps of removal of undesirable feed materials and sizing can be conducted in any order, i.e. either step can be conducted first and the other step conducted second. The remaining plastic mixture 101 is passed to an optional washing process 110 wherein the plastic mixture may be washed for example by treatment with a wash solution 112 to remove unwanted materials such as dirt, or labels, or coatings, or the like, to produce washed plastic mixture 111 and used solution 113. The plastic mixture 111 is passed to pyrolysis reactor 120. Optionally a vapor phase co-reactant comprising H2, CO, or olefins, or some combination of these, or a recycle stream can be fed directly to the pyrolysis reactor 120 or the catalytic pyrolysis reactor 140 (not shown). In the pyrolysis reactor 120 the mixture is heated to a temperature to decompose the plastics into a product mixture comprising a combination of vapor, solid and liquid phases. Without separation, at least a portion of the raw pyrolysis product mixture 121 is passed to catalytic reactor 140 while maintaining the temperature of the pyrolysis product mixture at least at the temperature at which it left the pyrolysis reactor 120. The pyrolysis product mixture 121 is passed to a hot catalytic reactor 140 that is charged with an aromatization catalyst effective at converting paraffins, or olefins, or both to aromatics, shown as catalytic product 141. A portion of the catalyst 142 is continuously withdrawn from reactor 140, or can be separated from the product 141, or both, and passed to catalyst regenerator 150. In catalyst regenerator 150 the catalyst is oxidized by treatment with a source of oxygen such as air 151 and the regenerated catalyst 143 is returned to reactor 140 and the combustion product gases 152 are vented or used to provide heat to the pyrolysis reactor or reactors. Catalytic product 141 can be separated into components such as ethylene, propylene, butenes, C1-C5 paraffins, benzene, toluene, xylenes, naphthalene, and other fractions in a separation scheme using conventional separation techniques. A portion of the materials separated from catalytic product stream 141 can be recycled to pyrolysis reactor 120 or catalytic pyrolysis reactor 140, or both.

FIG. 2 presents a schematic of another embodiment of the inventive process for converting plastic waste to olefins and aromatics. A mixture of plastics 10 is introduced into an optional feed system 100 that prepares the plastic mixture for introduction into the process by, for example, removing undesirable feed materials 102 such as metal, minerals, halogenated materials, and the like, or sizing the material to the desired size range, or both. The steps of removal of undesirable feed materials and sizing can be conducted in any order, i.e., either step can be conducted first and the other step conducted second. The remaining plastic mixture 101 is passed to an optional washing process 110 wherein the plastic mixture may be washed for example by treatment with a wash solution 112 to remove unwanted materials such as dirt, or labels, or coatings, or the like, to produce washed plastic mixture 111 and used solution 113. The prepared plastic mixture 111 is passed to thermal treatment reactor 115 with optional co-reactant 122 such as a heat transfer medium or getter or the like, where the mixture is heated to an intermediate temperature to partially decompose the plastics, e.g., decompose PVC or PVDC to release HCl, or decompose another halogenated polymer to release HCl, HBr, or HI, or release vapors such as NH₃, H₂O, or the like. An optional sweep gas 124 such as H₂O, N₂, Ar, CO₂, or some combination thereof is fed to thermal treatment reactor 115 to aid in the removal of vapors produced therein which are exhausted through exit port 125. With or without a sweep gas, the vapor 125 can be treated to capture or neutralize HCl and toxic materials prior to release or transfer to water treatment. The vapor 125 typically comprises at least 60% or at least 80% H₂O, and may contain HCl, halogenated carbon compounds, and other species more volatile than molten polymers. The condensed phases 126 are passed to pyrolysis reactor 120 where they are heated to decompose into a product mixture comprising a combination of solid, liquid, and vapor phases. While maintaining the temperature of the pyrolysis product mixture at least at the temperature at which it left pyrolysis reactor 120, the raw product mixture 121 is passed to a hot catalytic reactor 140 that is charged with an aromatization catalyst effective at converting paraffins, or olefins, or both to aromatics, shown as catalytic product 141. A portion of the catalyst 142 can be continuously withdrawn from reactor 140, or can be separated from the product 141, or both, and passed to catalyst regenerator 150. In catalyst regenerator 150 the catalyst is oxidized by treatment with a source of oxygen such as air 151 and the regenerated catalyst 143 is returned to reactor 140 and the combustion product gases 152 are vented or used to provide heat to the pyrolysis reactor or reactors. Catalytic product 141 can be separated into components such as ethylene, propylene, butenes, C1-C5 paraffins, benzene, toluene, xylenes, naphthalene, and other fractions in a separation scheme using conventional separation techniques. A portion of the materials separated from catalytic product stream 141 can be recycled to pyrolysis reactor 120 or catalytic pyrolysis reactor 140, or both

Combustible gases such as methane, ethane, propane, butanes, CO and H2, optionally, can be recovered from vapor stream 125 or recovered from the gases produced in the catalytic pyrolysis in the fluidized bed reactor. Combustible gases can provide heat for the process. Heat in reactors 115 or 120 may also be provided by pressure/friction and/or other heat sources such as resistive or inductive heating.

When recycled polymeric materials are used, impurities may optionally be removed from the feed composition prior to being fed to the reactor, e.g., by an optional separation step such as 100 in FIG. 1 or FIG. 2. In some instances, the separation step may include mechanical separation, sink/float separation, air elutriation, or other known separation processes, preferably in an automated mode. In some instances, the particle size of the solid polymer feed composition may be reduced in a size reduction system as part of 100 prior to passing the feed to the thermal treatment reactor or pyrolysis reactor. In some embodiments, the average diameter of the reduced size feed composition exiting the size reduction system may comprise no more than about 50%, not more than about 25%, no more than about 10%, no more than about 5%, no more than about 2% of the mass average diameter of the feed composition fed to the size reduction system. The feed mixture may comprise plastics mixtures in which at least 85% by mass, or at least 90% by mass, or at least 95% by mass of the particles pass through a 0.25 inch (0.6 cm), or 0.5 inch (1.2 cm), or 1.0 inch (2.5 cm), or 1.5 inch (3.7 cm), or 2 inch (5.0 cm), or 4 inch (10.0 cm) screen. Average diameter (size) can be determined by sieving through mesh (screen). Large-particle feed material may be more easily transportable and less difficult to process than small-particle feed material. On the other hand, in some cases it may be advantageous to feed small particles to the reactor. The use of a size reduction system allows for the transport of large-particle feed between the source and the process, while enabling the feed of small particles to the reactor.

The feed materials suitable for use in the invention can comprise all types of polymeric materials including polyethylene (PE), polypropylene (PP), polyacetylene, polybutylene, polyolefins, polyethylene terephthalate (PET), polybutylene terephthalate, polyester, copolyesters, polycarbonate, polyurethanes, polyamides, polystyrene (PS), polyacetal, epoxies, polycyanurates, polyacrylics, polyurea, vinyl esters, polyacrylonitrile, polyamide, polyurethane, polyethers, polycarbonates, poly(oxides), poly(sulfides), polyarylates, polyetherketones, polyetherimides, polysulfones, polyurethanes, polyvinyl alcohol, polyvinylchloride (PVC), polyvinyl dichloride (PVDC), polyvinyl acetate, nylon, copolymers such as ethylene-propylene, acrylonitrile-butadiene-styrene (ABS), nitrile rubber, natural and synthetic rubber, tires, styrene-butadiene, styrene-acrylonitrile, styrene-isoprene, styrene-maleic anhydride, ethylene-vinyl acetate, nylon 12/6/66, filled polymers, polymer composites, plastic alloys, other polymeric materials, and polymers or plastics dissolved in a solvent, whether obtained from polymer or plastic manufacturing processes as waste or discarded materials, post-consumer recycled polymer materials, materials separated from waste streams such as municipal solid waste, and polymers produced by polymerization of monomers, such as, for example, dienes, olefins, styrenes, acrylates, acrylonitrile, methacrylates, methacrylonitrile, diacids and diols, lactones, diacids and diamines, lactams, vinyl esters, block copolymers thereof, and alloys thereof; thermoset polymers such as, for example, epoxy resins; phenolic resins; melamine resins; alkyd resins; vinyl ester resins; unsaturated polyester resins; crosslinked polyurethanes; polyisocyanurates; crosslinked elastomers, including but not limited to, polyisoprene, polybutadiene, styrene-butadiene, styrene-isoprene, or some combination of these. The invention includes subcombinations of these materials, as desired, or as available from a particular location; the invention can be described as comprising one or any combination of these materials.

In any of the methods, the thermal treatment reactor 115 or pyrolysis reactor 120, or more than one of these, can be a moving bed reactor wherein the feed material is impelled along the length of the reactor by mechanical or gravitational means or both mechanical and gravitational means. Typical examples of reactors suitable for the thermal treatment reactor 115 or pyrolysis reactor 120, include a 1-screw extruder, 2-screw extruder, auger reactor, rotating kiln reactor, or stepped grate reactor. The pyrolysis reactor may have multiple heating zones with successively higher temperatures in later zones. The pyrolysis reactor can be fitted with a gas outlet at an area of the reactor where the temperature of the materials in the reactor is less than 300° C. or between 250° C. and 300° C. to allow for the removal of products produced at low temperatures such as steam, HCl, NH₃, or other materials from the reactor. A separating screen is fitted within the pyrolysis reactor immediately downstream of the gas outlet to at least partially prevent gases evolved at low temperature from passing along with the molten and solid materials into the hotter portions of the reactor. A gas inlet for the introduction of hot inert or recycle gas such as a gas comprising any of CH₄, H₂, CO, CO₂, and C2-C4 paraffins or olefins, or a mixture, can be fitted immediately downstream of the gas vent and optional screen.

Optionally, solid co-reactants 122, such as CaO, MgO, hydrotalcites, activated carbon, or zeolites, or some combination of these, that trap or remove undesirable components can be fed to thermal treatment reactor 115 and separated therefrom by filtration through a screen.

Where an auger reactor is utilized for thermal treatment or pyrolysis, the helical augers that optionally have different pitch dimensions at different portions of the auger in order to adjust the velocity of the condensed phases from the entry to the exit of the reactor. The flight thickness and shaft diameter may also be of variable dimension along the length of the auger in order to control the flow velocity of the vapor and condensed phases. Augers with paddles, or cuts, or folded flights are also envisioned as within the scope of the invention.

A rotating kiln reactor can be utilized for thermal treatment or pyrolysis. A kiln cylinder can be fitted with lifters, such as helical lifters attached to the cylinder wall or tabular lifters, folded lifters, or segmented lifters extending from the cylinder wall. A rotating kiln reactor as envisioned herein can also be inclined either up or down towards the exit end of the kiln depending on the desired residence time and flow velocity desired for the condensed phases within the kiln, thus taking advantage of gravity to control residence time of the condensed phases. It is also envisioned that the rotation rate of the rotating kiln reactor can be adjusted as desired, for example between 20 revolutions per minute to 0.2 revolution per minute depending on the nature of the feed mixture and the co-reactant added in order to provide thorough mixing and high heat transfer. A rotating kiln reactor as envisioned may be heated externally by the combustion of waste process gases such as CH₄, C2-C4 paraffins, H₂, CO, and the like recycled from the product separation or natural gas or electrically.

In any of the embodiments, the temperature profile within the pyrolysis reactor can range from a lower temperature near the feed entry port to a higher temperature at the exit port or ports. For example, the range of temperatures can be from 20° C. to 225° C., such as 20 to 100° C., or 20 to 50° C., at or near the inlet port, and the range of temperatures at the high-temperature exit port can be from 300° C. to 700° C., such as from 325 to 650° C., from 350 to 600° C., or from 350 to 575° C.

A solid co-reactant fed to the thermal treatment reactor can optionally be transferred to a combustion regenerator wherein the carbonaceous materials are reacted with air and at least a portion of the hot solid co-reactant material is returned to the thermal treatment reactor. The hot flue gas exiting the solid co-reactant regenerator can be passed to a catalyst heater to heat the catalyst for the catalytic pyrolysis reactor.

After leaving the thermal treatment reactor 115, the raw product preferably does not contact any cool surfaces that could condense products, and the surfaces are preferably maintained at a temperature of at least 300° C., at least 325, or at least 350° C. or within 25 or 50° C. of the temperature exiting the reactor 115. Preferably, in any of the embodiments, the temperature of the mixture is maintained at a temperature at least 2° C., or at least 3° C., or at least 5° C., or at least 10° C. higher than the temperature of the mixture at the exit end of the thermal treatment reactor.

In any of the inventive aspects, the catalytic reactor 140 can be a fluidized bed reactor; wherein the catalyst is a solid catalyst and the step of catalytically pyrolyzing comprises pyrolyzing in the presence of the solid catalyst in a fluidized bed reactor to produce a fluid product stream 141 and used catalyst with coke 142; and wherein at least a portion of the used catalyst with coke is transferred to a regenerator 150 where the coke is reacted with oxygen or air to form hot regenerated catalyst, and returning at least a portion of the hot regenerated catalyst 143 to the fluidized bed reactor, wherein heat from the hot regenerated catalyst provides energy to the step of catalytic pyrolyzing. In any of the methods the vapors exiting the catalytic pyrolysis reactor can be passed through an optional solids separation device such as a cyclone or screen to remove entrained solids. These entrained solids can be passed to the catalyst regenerator, or at least a portion can be returned to the catalytic pyrolysis reactor, or discarded, or some combination of these.

In any of the methods, the step of catalytically pyrolyzing may comprise pyrolysis in the presence of a fluid bed catalyst. The catalytic pyrolysis reactor may comprise a fluidized bed, circulating bed, bubbling bed, or riser reactor operating at a temperature in the range from 300° C. to 800° C., from 350° C. to 750° C., from 400° C. to 700° C., from 450° C. to 650° C., from 500° C. to 600° C. or from 525° C. to 575° C. The residence time of the vapors in the catalytic pyrolysis can be from 1 second to 480 seconds, from 1 second to 240 seconds, from 2 seconds to 60 seconds, from 3 seconds to 30 seconds, or from 4 seconds to 15 seconds. The pressure of the catalytic pyrolysis reactor can be at least 0.1 MPa (1 bar), at least 0.3 MPa (3 bar), or at least 0.4 MPa (4 bar), or from 0.1 to 2.0 MPa (1 to 20 bar), from 0.1 to 1.0 MPa (1 to 10 bar), or from 0.3 to 0.8 MPa (3 to 8 bar), preferably from 0.4 to 0.6 MPa (4 to 6 bar); pressures are absolute pressures.

Design and conditions of the fluidized bed catalytic reactor can be those conventionally known. A fluidization gas may be needed at start-up; during steady-state operation, fluidization gas may comprise a portion of vapor separated from stream 126 that, optionally, can be piped into the bottom of the fast catalytic pyrolysis fluidized bed reactor. Recycle gas from the process may be used as fluidizing gas. The fluidization gas can comprise H₂, CO, CO₂, H₂O, C1-C4 paraffins or olefins or both, N₂, Ar, He, or a recycle stream, or some combination thereof.

For catalytic pyrolysis, useful catalysts include those containing internal porosity selected according to pore size (e.g., mesoporous and pore sizes typically associated with zeolites), e.g., average pore sizes of less than 10 nm, less than 5 nm, less than 2 nm, less than 1 nm, less than 0.5 nm, or smaller. In some embodiments, catalysts with average pore sizes of from 0.5 to 10 nm may be used. In some embodiments, catalysts with average pore sizes of between 0.5 and 0.65 nm, or between 0.59 and 0.63 nm may be used. In some cases, catalysts with average pore sizes of between 0.7 and 0.8 nm, or between 0.72 and 0.78 nm may be used.

The catalyst composition particularly advantageous in the catalytic pyrolysis fluidized bed reactor of the present invention comprises a crystalline molecular sieve characterized by an SAR (silica to alumina, SiO₂:Al₂O₃ mass ratio) greater than 12, or from 12 to 240, and a CI (constraint index) from 1 to 12. Non-limiting examples of these crystalline molecular sieves are those having the structure of ZSM-5, ZSM-11, ZSM-12, ZSM-22, ZSM-23, ZSM-35, ZSM-48, ZSM-50, or combinations thereof. As an embodiment, the catalyst composition comprises a crystalline molecular sieve characterized by an SAR from greater than 12 to 240 and a CI from 5 to 10, such as, for example, molecular sieves having the structure of ZSM-5, ZSM-11, ZSM-22, ZSM-23 or combinations thereof. The method by which CI is determined is described more fully in U.S. Pat. No. 4,029,716, incorporated herein by reference for details of the method.

The molecular sieve for use herein or the catalyst composition comprising same may be thermally treated at high temperatures. This thermal treatment is typically performed by heating at a temperature of at least 370° C. for at least 1 minute and generally not longer than 20 hours (typically in an oxygen containing atmosphere, preferably air). While subatmospheric pressure can be employed for the thermal treatment, atmospheric pressure is desired for reasons of convenience. The thermal treatment can be performed at a temperature up to about 925° C. The thermally treated product is particularly useful in the present process.

For the catalyst compositions useful in this invention, the suitable molecular sieve may be employed in combination with a support or binder material such as, for example, a porous inorganic oxide support or a clay binder. Non-limiting examples of such binder materials include alumina, zirconia, silica, magnesia, thoria, titania, boria, and combinations thereof, generally in the form of dried inorganic oxide gels and gelatinous precipitates. Suitable clay materials include, by way of example, bentonite, kieselguhr, and combinations thereof. The relative proportion of suitable crystalline molecular sieve of the total catalyst composition may vary widely with the molecular sieve content ranging from 30 to 90 percent by weight and more usually in the range of 40 to 70 percent by weight of the composition. The catalyst composition may be in the form of an extrudate, beads or fluidizable microspheres.

The molecular sieve for use herein or the catalyst composition comprising it may have original cations replaced, in accordance with techniques well known in the art, at least in part, by ion exchange with hydrogen, or hydrogen precursor cations, or non-noble metal ions of Group VIII of the Periodic Table, i.e. nickel, iron or cobalt, or zinc, or gallium, or combinations thereof.

A portion of the vapor products from the catalytic pyrolysis process can be fed to a condenser where it is cooled to produce condensed materials. A portion of the condensed materials can be separated into fractions and at least a portion of the condensed material is recycled to the pyrolysis reactor or the catalytic pyrolysis reactor. The condensed material can be separated into fractions by distillation and at least a portion of the fraction boiling above 300° C. or boiling in the range 300 to 800° C. is recycled to the pyrolysis or catalytic pyrolysis reactor. The condensed material can be separated into fractions by distillation and at least a portion of the paraffins, olefins, or aromatics or their combination that contain more than 7 carbon atoms recycled to the pyrolysis or catalytic pyrolysis reactor.

In processes in which catalyst from the catalytic pyrolysis is regenerated, heat is generated by the oxidation of coke, char, and other materials in a catalyst regenerator for use in the process, or for conversion to electricity for export. In one set of embodiments, an oxidizing agent is fed to the regenerator via a stream shown as 151 in FIG. 1. The oxidizing agent may originate from any source including, for example, a tank of oxygen, atmospheric air, steam, among others. In the regenerator, the catalyst is re-activated by reacting the catalyst with the oxidizing agent and heat is generated. A solid mixture comprising deactivated catalyst may comprise residual carbon and/or coke as well as coke or char from the process, which may be removed via reaction with the oxidizing agent in the regenerator. In some embodiments a portion of the gaseous products from the catalytic pyrolysis process is fed to the catalyst regenerator to be combusted with the solid materials. The gaseous products may be first separated into an olefin rich stream and an olefin poor stream and at least a portion of the olefin poor stream may be fed to the catalyst regenerator. The regenerator in FIG. 1 comprises a vent stream 152 which may include regeneration reaction products, residual oxidizing agent, etc.

In some embodiments of the process at least a portion of the solid materials 123 that are removed from thermal treatment reactor 115 may be recycled to the feed of thermal treatment reactor 115 as a portion of the optional co-reactant 122. In some embodiments of the process, the optional co-reactant 122 may comprise solid materials that react with sulfur or nitrogen compounds to trap the sulfur or nitrogen species in the solid phase. The solid materials in the optional co-reactant 122 can comprise one or more materials chosen from among agricultural lime, calcium oxide, calcium hydroxide, magnesium oxide, magnesium hydroxide, limestone, or hydrotalcites, activated carbon, or zeolite, or some combination thereof.

Waste plastics are collected locally at numerous facilities that each handle small amounts of plastics in any one day, either as part of general waste (municipal solid waste) or a separate recycling stream. In either case, most of the material ends up among the 25 million tons of plastics that are sent to one of more than 2,600 landfills in the USA each year. The amount of waste plastic available at any one site is typically on the order of only a few tens of metric tonnes per day.

A chemical plastics recycling plant includes feed handling, cleaning, processing (e.g. pyrolysis and catalytic pyrolysis), recovery, separations, and purifications operations. The cost of the separations and purification facilities often constitute 35-50% of the capital cost of a complete facility.

Plants that have small capacities are more expensive on a per-tonne-of-product basis than larger plants due to the lack of the economy of scale. One way to take advantage of economies of scale for the separations and purifications functions is to network together several plants that produce crude mixtures of liquid products of a similar composition, and send the crude mixtures to a refinery or other central processing facility for separation and purification into chemical grade materials.

Several commercial plastics upgrading processes produce liquid products that are not ready for separation and purification due to the presence of long chain hydrocarbons and olefins that must be further upgraded by hydroprocessing such as in a hydrocracker, or hydrotreater, or steam cracker or some combination of these. Hydroprocessing requires a source of hydrogen, whereas the products of the present process require little or no hydrogen for upgrading and can be separated and purified without hydroprocessing. In addition, heating the feeds for hydroprocessing requires energy addition that cannot be fully recovered, which is avoided with the current invention. Hydroprocessing is exothermic, so considerable heat is released in the process, which can cause problems with heat removal if the level of olefins is too high for the heat removal capabilities of the system.

A benefit of the invention of a two-step process for upgrading plastics by pyrolysis followed by catalytic pyrolysis is the ability to produce a liquid product that is suitable for combining with conventional refinery streams such as the product of a steam cracker or hydrocracker, and which can be more readily stored and transported compared to gaseous products, for separation and purification at a larger facility. This means that a crude liquid product stream made from recycled plastic by the inventive process can be produced in a separate location from the product purification system, and this “distributed processing” scheme may be advantageous as separation and purification costs can be minimized for small scale regional facilities.

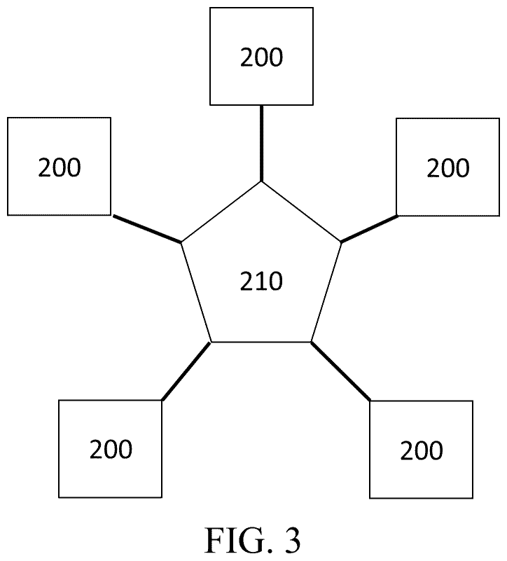

FIG. 3 shows an embodiment of the invention in which five plastics upgrading units 200 are connected to feed a single product separation and purification facility 210 in a hub and spoke system.

In some embodiments a system for upgrading waste plastics comprises the first pyrolysis reactor and catalytic fluidized bed reactor that together form one spoke of a ‘hub-and-spoke’ network for producing refined chemical intermediates such as benzene, toluene, xylenes, p-xylene, m-xylene, o-xylene, BTX (a mixture of benzene, toluene, and xylenes), C6-C20 paraffins and olefins, ethylene, propylene, naphthalene, or others. or some combination of these, where each of the more than one plastics upgrading sites (the spokes) produces condensed phase products that are sent to a central processing facility (the hub) for separation and purification into product streams. In some embodiments of the system the number of plastics upgrading facilities that can be in a network feeding a single central separation and purification facility can be at least 2, at least 3, at least 5, at least 7, at least 10, or at least 15, or from 2 to 20, from 3 to 10, or from 5 to 10 plastics upgrading facilities. The total crude product mixture prepared at the plastics upgrading facilities that is introduced into a central separation and purification facility can be at least 20, at least 50, at least 100, at least 150, or at least 200 metric tons per day, or from 20 to 500, from 30 to 200, or from 50 to 150 metric tons per day of crude product mixture.

In some embodiments a system for upgrading plastics comprising the first pyrolysis reactor as one ‘spoke’ in a ‘hub and spoke’ network for producing refined chemical intermediates such as benzene, toluene, xylenes, p-xylene, m-xylene, o-xylene, BTX (a mixture of benzene, toluene, and xylenes), C6-C20 paraffins and olefins, ethylene, propylene, naphthalene, or others. or some combination of these, where each of the more than one plastics upgrading sites (the spokes) produces condensed phase products that are sent to a central processing facility (the hub) that includes a central fluidized bed catalytic process plant (Plas-TCat™) and separation and purification into product streams. In some embodiments of the system the number of plastics pyrolysis facilities that can be in a network feeding a single central catalytic upgrading, separation, and purification facility can be at least 2, at least 3, at least 5, at least 7, at least 10, or at least 15, or from 2 to 20, from 3 to 10, or from 5 to 10 plastics pyrolysis facilities. The total crude product mixture prepared at the plastics pyrolysis facilities that is introduced into a central catalytic upgrading, separation, and purification facility can be at least 20, at least 50, at least 100, at least 150, or at least 200 metric tons per day, or from 20 to 500, from 30 to 200, or from 50 to 150 metric tons per day of crude product mixture.

### Examples 1 Through 10

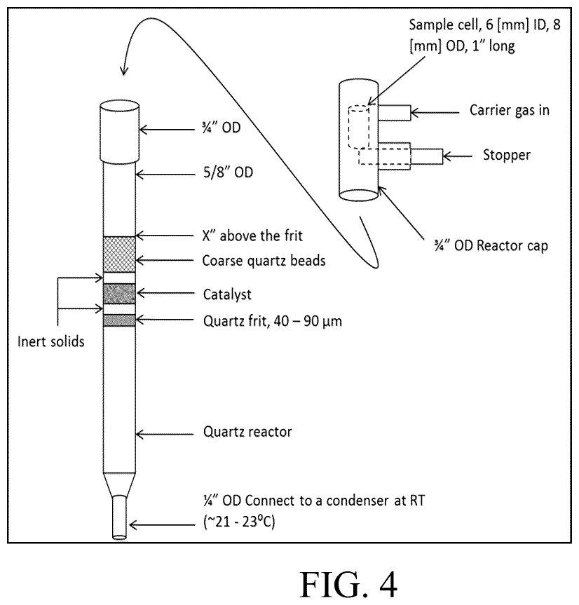

The drop-tube reactor for two-step chemical conversion of plastics without separation between the pyrolysis and catalytic pyrolysis steps comprises a quartz reactor tube (ACE Glass) containing a quartz frit (40-90 μm) fused into the center of the tube. FIG. 4 shows the configuration of the drop-tube reactor. A sample cell (10 mm OD, 8 mm ID, 25 mm length, quartz, made by TGP) is used to contain the feedstock using two pieces of quartz wool (TGP). As illustrated in FIG. 4, the sample cell was placed in a reactor cap (borosilicate, ACE Glass) and was held by a stopper (¼ inch (6 mm) aluminum rod, McMaster). The reactor cap and the quartz reactor were then assembled and installed onto the fixed-bed reactor system. The bottom of the reactor was connected to a condenser (borosilicate) filled with perforated stainless steel packing (ACE Glass) immersed in an ice-water bath (0° C.). A heating mantle was applied between the reactor bottom and the condenser top to prevent any condensation before the condenser. During the reaction, the heating mantle was set at 210° C.

In the reactor, a small sample of ZSM-5 catalyst (1.5 g) was placed on top of the quartz frit. Feedstock (100 mg for each run) was sealed in a sample cell with the quartz wool. The catalyst/feedstock weight ratio was about 15. Prior to dropping the contents of the sample cell into the reactor, the catalyst was calcined at 550° C. under 100 mL/min air flow for 20 min (ramping rate=12° C./min). After calcination, the reactor was cooled to reaction temperature (500° C.). During the cool-down, the condenser was filled with 10 mL of solvent (ethyl acetate for plastics conversion, and acetone for biomass conversion) and held for 10 min for temperature lineout. The reactor system was then purged with helium flow at 75 mL/min for 20 min to remove air and to purge the gas collection lines. The sample cell containing the feed material was dropped into the reactor by pulling out the stopper rod to initiate the reaction.

A hold period of 10 min allowed the reaction to complete. Gas products, consisting mostly of permanent gases and C₁-C₃ olefins and paraffins were collected in a gas bag. Liquid products (mostly C₄₊) were collected in the condenser. After reaction the temperature was increased to 650° C. without gas flow. Solid products, including coke and char remaining in the reactor, were then burned at 650° C. for 10 min under 50 mL/min air flow. The gas products during burning were collected in a second gas bag. An additional 3 mL of solvent was added to the condenser to extract any products remaining on the top of the condenser. All of the liquid in the condenser was then transferred to a 20 mL sample vial. A weighed amount of internal standard (dioxane, typically 3000-5000 mg, Sigma-Aldrich) was added to the sample vial. The condenser was washed with acetone and was dried in a drying oven. It is noted that a small amount of liquid was retained in the condenser due to holdup on the packing. Therefore, the weight of the condenser with and without liquid products was measured to obtain the total amount of liquid products. Liquid samples were analyzed by a GC-FID (gas chromatograph with flame ionization detector from Shimadzu 2010Plus) for hydrocarbons and oxygenates. Gas bag samples were analyzed using an Agilent GC 7890B gas chromatograph.

The results of the experiments for various feeds are presented in TABLE 2. The balances of the products unaccounted for in TABLE 2 comprise water, inert solids, and minor components not readily recovered for combustion.

Examples 1 through 10 show that the two-step pyrolysis followed by catalytic pyrolysis without an intervening separation step produces high yields of olefins and aromatics from plastics. The yield of olefins is at least 2% in all cases, and the yield of BTX is at least 10.08% in all cases. Examples 1, 2, 3, 4, and 9 show that for polymers that do not contain fillers (tires) or heteroatoms (PET, nylon), the yield of BTX is at least 32.88%, the yield of olefins is at least 5.58%, and the yield of coke and char is less than 5%, and often less than 2% of the mass of the feed. The yield of olefins for two-step pyrolysis/catalysis for polyolefins (Examples 1, 2, 3, and 4) is at least 10.43%, and the yield for linear, non-branched polyolefins (Examples 1, 2, and 3) is at least 17.25%. The yield of aromatics for two-step pyrolysis/catalysis for polyolefins (Examples 1, 2, 3, and 4) is at least 32.88%, and the yield for linear, non-branched polyolefins (Examples 1, 2, and 3) is at least 45.6%.

TABLE 2
Products of catalytic pyrolysis of various materials with ZSM-5
catalyst in drop tube experiments. All values are weight percent.

| | | | Other | Coke | | | | | | | |
|---|---|---|---|---|---|---|---|---|---|---|---|
| | | | C5 + | and | | | | | | | |
| Ex | Feed | BTX | Liquid | Char | Olefins | Paraffins | H2 | CO | CO2 | Ash | Total |
| 1 | Polyethylene (PE) | 52.8 | 3.5 | 0.9 | 18.1 | 16.4 | 2.6 | 0.3 | 0.3 | 0 | 94.8 |
| 2 | Polypropylene (PP) | 45.6 | 4.0 | 0.7 | 21.5 | 16.5 | 2.4 | 0 | 0.2 | 0 | 90.8 |
| 3 | High Density | 53.05 | 3.47 | 1.05 | 17.25 | 18.53 | 2.7 | 0 | 0.21 | 0 | 96.3 |
| | Polyethylene | | | | | | | | | | |
| | (HDPE) | | | | | | | | | | |
| 4 | Isoprene | 32.88 | 10.03 | 1.29 | 10.43 | 5.89 | 1.29 | 0 | 0.28 | 0 | 62.1 |
| 5 | Tire Sidewall | 14.94 | 3.13 | 21.62 | 8.25 | 2.08 | 0.9 | 0.56 | 0.95 | 7.0 | 59.4 |
| 6 | Tire Tread | 13.9 | 2.99 | 20.37 | 7.53 | 2.12 | 0.94 | 0.25 | 0.8 | 7.0 | 55.9 |
| 7 | PET | 23.73 | 3.92 | 17.76 | 4.98 | 1.16 | 0.38 | 7.65 | 34.13 | 0 | 93.7 |
| 8 | PET Clothing | 23.06 | 4.12 | 16.96 | 2.17 | 1.3 | 0.38 | 7.37 | 31.67 | 0 | 87.0 |
| 9 | Polystyrene (PS) | 38.51 | 36.29 | 4.13 | 5.58 | 1.49 | 0.48 | 0 | 0.33 | 0 | 86.8 |
| 10 | Nylon | 10.08 | 6.2 | 10.0 | 16.57 | 1.23 | 1.2 | 3.72 | 4.19 | 0 | 53.2 |

1. A method for producing olefins and aromatics comprising;
feeding a stream comprising plastics to a first pyrolysis reactor;
anaerobically pyrolyzing the stream in the first pyrolysis reactor at a temperature between 250 and 300 and holding at that temperature range while vapors are removed;
and further pyrolyzing at higher temperature in the first pyrolysis reactor to prepare a first product mixture;
without separating, passing the first product mixture produced in the first pyrolysis reactor to a second pyrolysis reactor that comprises a fluidized bed reactor fitted with a catalyst;
catalytically reacting the product mixture in the fluidized bed reactor to form a catalytic pyrolysis product mixture; and
recovering olefins, or aromatics, or some combination thereof from the pyrolysis product mixture.

2. The process of claim 1 wherein the first product mixture produced in the first pyrolysis reactor is passed to the second pyrolysis reactor at a temperature above 350° C. without cooling.

3. (canceled)

4. The process of claim 1 wherein the first pyrolysis reactor comprises a feed inlet port and an exit port and the temperature in the pyrolysis reactor ranges from a lower temperature near the feed inlet port to a higher temperature at the exit port.

5. (canceled)

6. (canceled)

7. The process of claim 1 wherein the first pyrolysis reactor comprises two or more reactors in series.

8-12. (canceled)

13. The process of claim 1 wherein the catalyst in the fluidized bed reactor comprises a zeolite.

14. The process of claim 13 wherein the catalyst has a silica to alumina greater than 12, or from 12 to 240, and a CI (constraint index) from 1 to 12 or from 5 to 10.

15. (canceled)

16. The process of claim 13 wherein the catalyst comprises ZSM-5.

17-21. (canceled)

22. The process of claim 1 wherein a product vapor mixture from the fluidized bed catalytic reactor comprises at least 20 mass % BTX.

23. The process of claim 1 wherein a solid co-reactant is fed to the first pyrolysis reactor wherein the solid co-reactant fed to the first pyrolysis reactor comprises agricultural lime, or calcium oxide, calcium hydroxide, magnesium oxide, magnesium hydroxide, limestone, hydrotalcites, activated carbon, or zeolite, or other solid basic material, or some combination thereof.

24-27. (canceled)

28. The process of claim 1 wherein the temperature of the fluidized bed reactor is in the range from 300° C. to 800° C., from 350° C. to 750° C., from 400° C. to 700° C., from 450° C. to 650° C., from 500° C. to 600° C., or from 525° C. to 575° C.

29-32. (canceled)

33. The process of claim 1 wherein benzene, toluene, or xylenes are separated or recovered from the catalytic pyrolysis product mixture.

34. The process of claim 1 wherein at least a portion of the aromatic products in the catalytic pyrolysis product mixture is hydrogenated to produce naphthenes.

35. (canceled)

36. The process of claim 1 wherein the catalytic pyrolysis product mixture is subjected to a separation process to produce a stream of gases enriched in CH₄, CO, and H₂; and passing at least a portion of the stream of gases enriched in CH₄, CO, and H₂ to a regenerator where they are combusted.

37. (canceled)

38. (canceled)

39. The process of claim 1 wherein the feedstock comprises a mix of waste plastic chosen from among polyethylene terephthalate (PET), high density polyethylene (HDPE), polyvinyl chloride (PVC) or polyvinylidene (PVCD), low density polyethylene (LDPE), polypropylene (PP), polystyrene (PS), or mixed resins, or some combination thereof.

40-50. (canceled)

51. The process of claim 1 wherein a portion of the catalytic pyrolysis product mixture is fed to a condenser where it is cooled to produce condensed materials and a portion of the condensed materials is recycled to the first pyrolysis reactor.

52. The process of claim 51 wherein a portion of the condensed materials is separated into fractions and at least a portion of the condensed materials is recycled to the first pyrolysis reactor or the second pyrolysis reactor.

53. The process of claim 51 wherein the condensed materials is separated into fractions by distillation and at least a portion comprising paraffins, olefins, or aromatics or their combination that contain more than 7 carbon atoms is recycled to the first pyrolysis reactor or the second pyrolysis reactor.

54. (canceled)

55. The method of claim 1 wherein the step of further pyrolyzing at higher temperature is in the range of 350 to 600° C.

56. The method of claim 4 wherein the temperature in the second pyrolysis reactor is maintained at a temperature at least 5 C higher than the temperature at the exit port.
